(19) United States
(12) Patent Application Publication
(10) Pub. No.: US 2019/0314533 A1
(43) Pub. Date: Oct. 17, 2019

(54) ULTRAVIOLET STERILIZATION MODULE

(71) Applicant: POINT ENGINEERING CO.,LTD., Asan (KR)

(72) Inventors: Bum Mo AHN, Suwon (KR); Seung Ho PARK, Hwaseong (KR); Moon Hyun KIM, Ansan (KR)

(21) Appl. No.: 16/383,406

(22) Filed: Apr. 12, 2019

(30) Foreign Application Priority Data
Apr. 13, 2018 (KR) 10-2018-0043554

(51) Int. Cl. A61L 2/10; A61L 2/26

(52) U.S. Cl. CPC A61L 2/10; A61L 2209/12; A61L 2/26

(57) ABSTRACT

The present invention relates generally to an ultraviolet sterilization module for sterilizing a fluid using ultraviolet light. More particularly, the present invention relates to an ultraviolet sterilization module, configured such that an increase in amount of ultraviolet irradiation is achieved, thus efficiently sterilizing a fluid inside a light transmission member without requiring an increase in size of the light transmission member.

### CROSS REFERENCE TO RELATED APPLICATION

The present application claims priority to Korean Patent Application No. 10-2018-0043554, filed Apr. 13, 2018, the entire contents of which is incorporated herein for all purposes by this reference.

### BACKGROUND OF THE INVENTION

### Field of the Invention

The present invention relates to an ultraviolet sterilization module for sterilizing a fluid using ultraviolet light.

### Description of the Related Art

In general, sterilization refers to the complete elimination of all living organisms, including viruses, bacteria, fungi, microorganisms, mites, and the like. Common methods of sterilization include a physical method including a heating method for sterilizing an object by heating using heat or steam, and a chemical method for sterilizing an object using a sterilizing agent or a sterilizing gas.

In recent years, as the awareness of bacteria and the interest in health have increased among the general public, there has been a growing interest in devices or methods that can facilitate sterilization.

The physical method, which is generally used to eliminate viruses, bacteria, microorganisms, or mites, includes a method in which microorganisms or the like are eliminated by applying heat or steam having a high temperature to an object to be sterilized, is problematic in that fuels, a time to secure a high temperature, and the like are required and thus it takes a long time to sterilize. Additionally, the physical method is still problematic in that a user may be injured by heat or steam having a high temperature, which is difficult to handle. The chemical method using a sterilizing agent or a sterilizing gas is problematic in that the sterilizing agent is generally made of a chemical substance and thus a user may be exposed to toxic substances in the process of using the sterilizing agent or the sterilizing gas.

In an effort to solve the above problems, there has been developed a sterilization method using ultraviolet irradiation which is a physical sterilization method. Particularly, recently, an optical device such as a light emitting diode (LED) has been used to generate ultraviolet light, thus compensating for the above-mentioned disadvantages of requiring fuels, the time to secure a high temperature, and the like.

In regard to the above, Korean Patent No. 10-1824951 (hereinafter referred to as “Patent Document 1”) disclosed a ballast water sterilization device using an ultraviolet light-emitting diode (UV-LED).

The ballast water sterilization device disclosed in Patent Document 1 includes a housing in which a ballast water flows in and out, and an ultraviolet sterilization device for sterilizing microorganisms and the like existing in the ballast water flowing into the inside of the housing.

However, the ballast water sterilization device disclosed in Patent Document 1 has a structure in which upper and lower surfaces of a substrate of an optical module of the ultraviolet sterilization device have a linear shape and thus are difficult to attach directly to inner and outer surfaces of a protection tube having a curved surface.

Due to such a structure, the ultraviolet sterilization device has to be provided at the center of the interior of the housing, resulting that ultraviolet irradiation is performed only outwardly from the center of the interior of the housing in which the ballast water flows.

Therefore, an increase in size of the ultraviolet sterilization device is inevitable for increasing the amount of ultraviolet irradiation. However, increasing the size of the ultraviolet sterilization device causes a problem in that the amount of ballast water sterilized inside the housing may be rather significantly reduced.

In the foregoing description, the ballast water has been described by way of example, but it is not limited thereto.

Even when a fluid to be sterilized passes through a cylindrical fluid tube, and the fluid inside the fluid tube is to be sterilized, the same problem as above may occur.

Particularly, in the case where the optical module is manufactured separately from the fluid tube and provided near the wall of the fluid tube, the fluid tube and the optical module has to be located to be spaced apart from each other. This causes a problem in that a sterilization area may be reduced and an unsterilized area in which sterilization is not performed may be generated, resulting in degrading sterilization efficiency.

The foregoing is intended merely to aid in the understanding of the background of the present invention, and is not intended to mean that the present invention falls within the purview of the related art that is already known to those skilled in the art.

Documents of Related Art

(Patent document 1) Korean Patent No. 10-1824951

### SUMMARY OF THE INVENTION

Accordingly, the present invention has been made keeping in mind the above problems occurring in the related art, and an objective of the present invention is to provide an ultraviolet sterilization module, configured such that an increase in amount of ultraviolet irradiation is achieved, thus efficiently sterilizing a fluid inside a light transmission member without requiring an increase in size of the light transmission member.

In order to achieve the above objective, according to one aspect of the present invention, there is provided an ultraviolet sterilization module, including: a light transmission member having a flow passage that is formed therein and through which a fluid flows; and an optical device package provided on an outer surface of the light transmission member, wherein the optical device package includes: a unit substrate including first and second metal substrates that are bonded together on side surfaces thereof in a horizontal direction, and a vertical insulating layer provided between the first and second metal substrates so as to electrically insulate the first and second metal substrates; a cavity provided in an upper surface of the unit substrate; and an ultraviolet optical device mounted in the cavity, wherein an upper surface of the unit substrate is in surface contact with the outer surface of the light transmission member.

Furthermore, the light transmission member may have a cylindrical shape, and the unit substrate of the optical device package may have a first curved surface having the same curvature as a curvature of the cylindrical-shaped light transmission member.

Furthermore, the optical device package may be provided on an outer circumferential surface of the cylindrical-shaped light transmission member in a state where multiple unit substrates are connected to each other by junctions.

Furthermore, each of the junctions may have a groove formed in a lower surface of the unit substrate to be open downward.

Furthermore, the vertical insulating layer of the optical device package may be provided such that a longitudinal direction thereof is perpendicular to a central axis of the light transmission member.

Furthermore, the optical device package may be provided in plural, and multiple optical device packages may be configured such that multiple unit substrates of each of the optical device packages are connected to each other and arranged along a longitudinal direction of the cylindrical-shaped light transmission member.

Furthermore, the vertical insulating layers of the optical device packages may be provided such that longitudinal directions thereof are parallel to a central axis of the light transmission member.

The ultraviolet sterilization module according to the present invention as described above has the following effects.

Through provision of the first curved surface, the upper surface of the optical device package has a shape corresponding to the shape of the curved surface of the light transmission member. This makes it possible for the optical device package to be bonded to the light transmission member in surface contact therewith. Therefore, it is possible to prevent defective bonding, leakage of light, and the like that may be caused when a portion of the optical device package is not in contact with the light transmission member.

The optical device package has a configuration in which provision of a light transmission member bonded to the upper surface of an optical device package according to the related art is eliminated, and the surface of the optical device package that is to be in direct contact with the light transmission member having the curved surface is changed to the first curved surface. Therefore, it is possible to prevent the transmittance from being decreased due to the light transmission member of the optical device package according to the related art when light generated in the optical device is radiated, and also to prevent the optical device from being damaged due to penetration of foreign substances such as water into the cavity.

The multiple unit substrates of the optical device package are bonded to the light transmission member in a state of being rolled in a circular shape by the grooves, thus making it possible to easily achieve surface contact bonding to the curved surface of the light transmission member, and also to efficiently bond a large number of unit substrates to the light transmission member. Therefore, it is possible to achieve an increase in sterilization efficiency of the ultraviolet sterilization module.

The multiple unit substrates of the optical device package are connected to each other, thus making it possible to achieve a strong bonding with the light transmission member and thus to prevent the ultraviolet sterilization module from being damaged.

The first and second metal substrates of the optical device package provided on the light transmission member are disposed in predetermined orientations with respect to the vertical insulating layer, thus making it possible to easily achieve application of voltage to the first and second metal substrates.

### BRIEF DESCRIPTION OF THE DRAWINGS

The above and other objectives, features and other advantages of the present invention will be more clearly understood from the following detailed description when taken in conjunction with the accompanying drawings, in which:

FIG. 1 is a perspective view illustrating an ultraviolet sterilization module according to a first embodiment of the present invention;

FIG. 2 is a perspective view illustrating that an optical device is mounted on an optical device package of FIG. 1;

FIG. 3A is a cross-sectional view taken along line A-A′ of FIG. 2;

FIG. 3B is a cross-sectional view taken along line B-B′ of FIG. 2;

FIG. 4 is a perspective view illustrating a modified embodiment of the ultraviolet sterilization module according to the first embodiment of the present invention;

FIG. 5 is a perspective view illustrating an ultraviolet sterilization module according to a second embodiment of the present invention;

FIG. 6 is a perspective view illustrating that an optical device is mounted on an optical device package of FIG. 5;

FIG. 7A is a sectional view taken along an X-axis of the optical device package of FIG. 6;

FIG. 7B is a sectional view taken along a Y-axis of the optical device package of FIG. 6; and

FIG. 8 is a perspective view illustrating a first modified embodiment of the ultraviolet sterilization module according to the second embodiment of the present invention;

FIG. 9 is a perspective view illustrating a second modified embodiment of the ultraviolet sterilization module according to the second embodiment of the present invention; and

FIG. 10 is a perspective view illustrating an ultraviolet sterilization module according to a third embodiment of the present invention.

### DETAILED DESCRIPTION OF THE INVENTION

Contents of the description below merely exemplify the principle of the invention. Therefore, those of ordinary skill in the art may implement the theory of the invention and invent various apparatuses which are included within the concept and the scope of the invention even though it is not clearly explained or illustrated in the description. Furthermore, in principle, all the conditional terms and embodiments listed in this description are clearly intended for the purpose of understanding the concept of the invention, and one should understand that this invention is not limited to such specifically listed exemplar)/embodiments and conditions.

The above described objectives, features, and advantages will be more apparent through the following detailed description related to the accompanying drawings, and thus those of ordinary skill in the art may easily implement the technical spirit of the invention.

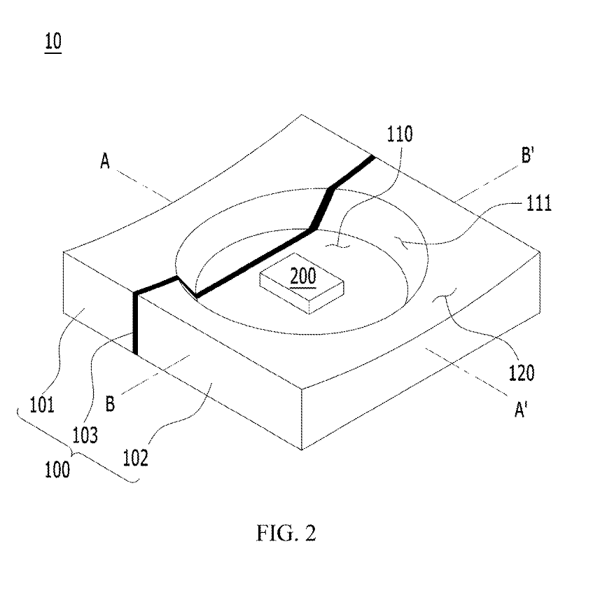

In the following description, the X-axis denotes an axis that connects the left side and the right side of an optical device package 10 of FIG. 2, and the Y-axis denotes an axis that is perpendicular to the X-axis and connects the front side and the rear side of the optical device package 10 of FIG. 2.

Therefore, a direction from the left side to the right side of the optical device package 10 of FIG. 2, that is, a direction in which line A-A′ is drawn is an X-axis direction, and a direction from the front side to the rear side, that is, line B-B′ is drawn is an Y-axis direction.

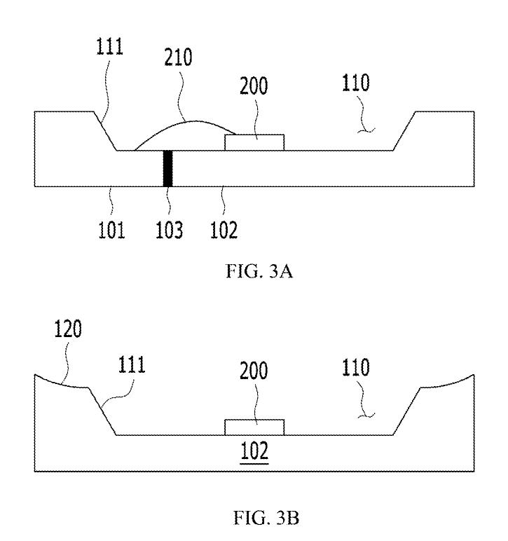

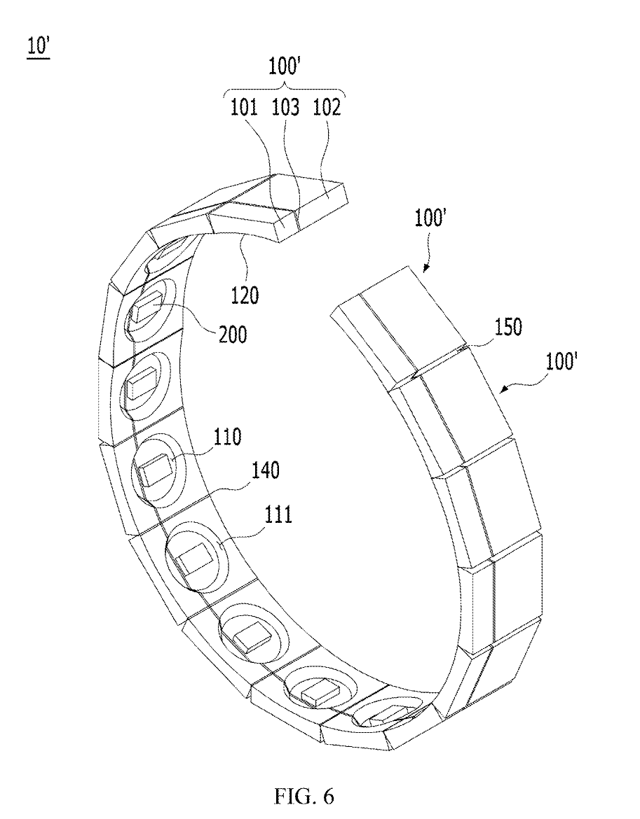

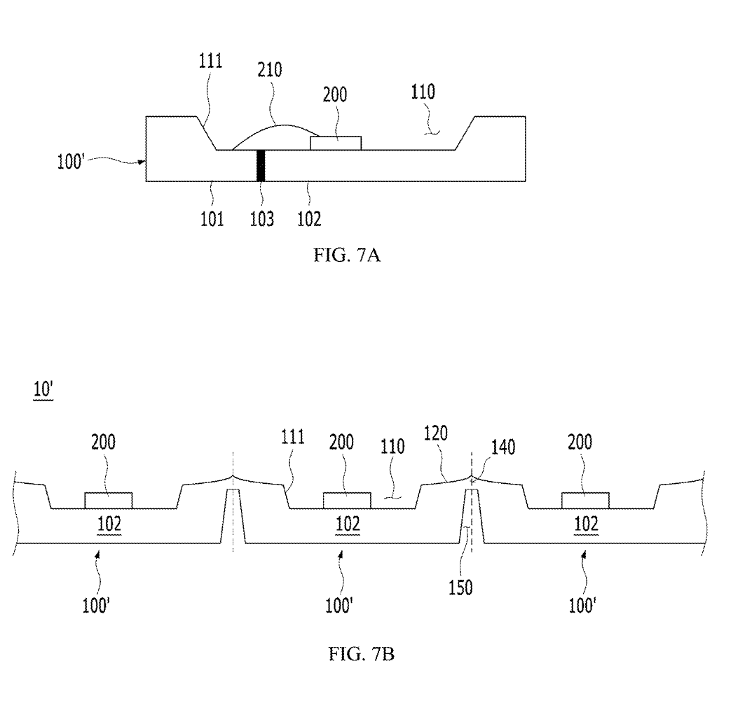

FIG. 2 is view in which a wire 210 of FIG. 3A is not illustrated, and FIG. 6 is a view in which a wire 210 of FIG. 7A is not illustrated.

Ultraviolet Sterilization Module 1 According to a First Embodiment of the Present Invention

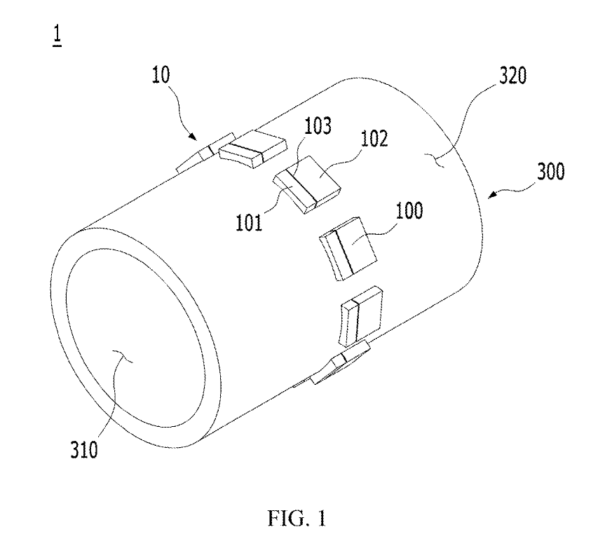

Hereinbelow, an ultraviolet sterilization module 1 according to a first embodiment of the present invention will be described with reference to FIGS. 1 to 3B.

FIG. 1 is a perspective view illustrating an ultraviolet sterilization module according to a first embodiment of the present invention, FIG. 2 is a perspective view illustrating that an optical device is mounted on an optical device package of FIG. 1, FIG. 3A is a cross-sectional view taken along line A-A′ of FIG. 2, and FIG. 3B is a cross-sectional view taken along line B-B′ of FIG. 2.

As illustrated in FIGS. 1 and 2, the ultraviolet sterilization module 1 according to the first embodiment of the present invention includes a light transmission member 300 having a flow passage 310 that is formed therein and through which a fluid flows, and an optical device package 10 provided on an outer surface of the light transmission member 300.

The light transmission member 300 has a cylindrical shape with the flow passage 310 formed therein and a curved surface 320. In this case, it is preferable that the curved surface 320 is the same in curvature as a first curved surface 120.

It is preferable that the light transmission member 300 is made of a material having excellent translucency such that light emitted from an ultraviolet optical device 200 of the optical device package 10 is transmitted without being scattered. For example, the light transmission member 300 is any one selected from among the group consisting of glass, quartz, and the like, but is not limited thereto.

The optical device package 10 is bonded to the light transmission member 300 such that an upper surface thereof, that is, an upper surface of a unit substrate 100 comes into surface contact with the outer surface of the light transmission member 300. In this case, as illustrated in FIG. 1, multiple optical device packages 10 are bonded to the outer surface, that is, an outer circumferential surface of the light transmission member 300 by being arranged at regular intervals along a circumferential direction of the light transmission member 300.

As illustrated in FIGS. 2, 3A, and 3B, each of the optical device packages 10 includes: the unit substrate 100 having first and second metal substrates 101 and 102 that are bonded together on side surfaces thereof in a horizontal direction, and a vertical insulating layer 103 provided between the first and second metal substrates 101 and 102 so as to electrically insulate the first and second metal substrates 101 and 102 from each other; a cavity 110 provided in the upper surface of the unit substrate 100; the first curved surface 120 provided on the upper surface of the unit substrate 100; and the ultraviolet optical device 200 mounted in the cavity 110.

The unit substrate 100 includes the first metal substrate 101, the second metal substrate 102, and the vertical insulating layer 103.

As illustrated in FIG. 3A, the first metal substrate 101 and the second metal substrate 102 are bonded together on a right surface and a left surface thereof, respectively, by the vertical insulating layer 103. Therefore, the first metal substrate 101, the vertical insulating layer 103, and the second metal substrate 102 are sequentially arranged from the left to the right.

The first metal substrate 101 and the second metal substrate 102 are made of a metal plate having excellent electrical conductivity and thermal conductivity. For example, the first metal substrate 101 and the second metal substrate 102 are made of any one selected from among the group consisting of aluminum, an aluminum alloy, copper, a copper alloy, iron, an iron alloy, and the like, but are not limited thereto.

Although not illustrated in the drawings, each of the first metal substrate 101 and the second metal substrate 102 has an electrode layer provided at a lower portion thereof. The electrode layer is made by plating of a metal material or the like and is connected to a circuit board or to a power source to apply voltage to the first and second metal substrates 101 and 102.

The vertical insulating layer 103 is vertically disposed between the first metal substrate 101 and the second metal substrate 102 and functions to electrically insulate the first metal substrate 101 and the second metal substrate 102 from each other and to bond the first metal substrate 101 and the second metal substrate 102 together.

The first metal substrate 101 and the second metal substrate 102 are electrically insulated from each other by the vertical insulating layer, whereby different voltages are applied to the first metal substrate 101 and the second metal substrate 102. The vertical insulating layer 103 is made of any one selected from among the group consisting of a conventional insulating sheet, benzocyclobutene (BCB), bismaleimide trizine (BT), polybenzoxazole (PBO), polyimide (PI), phenolicresin, epoxy, silicone, and the like, but is not limited thereto.

In the case where the first and second metal substrates 101 and 102 are made of aluminum or an aluminum alloy, the vertical insulating layer 103 includes an aluminum anodic oxide film formed by anodic oxidation of aluminum.

The cavity 110 is depressed downward in the upper surface of the unit substrate 100 so as to be located at the center of the unit substrate 100 and provides a space for mounting the ultraviolet optical device 200 therein.

The cavity 110 has a tapered bowl shape configured such that the width thereof decreases downward. Therefore, the cavity 110 is provided with an inclined surface 111, and the inclined surface 111 is inclined inwardly from the top of the unit substrate 100.

Due to the tapered bowl shape of the cavity 110, the lower width of the cavity 110 is smaller than the upper width thereof. The inclined surface 111 functions to reflect light generated in the ultraviolet optical device 200 mounted in the cavity 110. Therefore, when the ultraviolet optical device 200 is mounted in the cavity 110, light efficiency of light generated in the ultraviolet optical device 200 is further increased.

The cavity 110 having the above-described configuration is formed by forming or the like of the upper surface of the unit substrate 100. Therefore, the cavity 110 is depressed downwardly of the first and second metal substrates 101 and 102 by forming or the like of a part of an upper surface of the first metal substrate 101 and a part of an upper surface of the second metal substrate 102. Furthermore, the cavity 110 is formed by removing a part of an upper portion of the vertical insulating layer 103 disposed between the first and second metal substrates 101 and 102.

The first curved surface 120 is formed on the upper surface of the unit substrate 100 and functions to allow the upper surface of the unit substrate 100 of the optical device package 10 to come into surface contact with the curved surface 320 of the light transmission member 300 when the optical device package 10 is bonded to the light transmission member 300.

The first curved surface 120 is formed on the upper surface of the unit substrate 100, that is, on the upper surfaces of the first and second metal substrates 101 and 102 by forming or the like.

It is preferable that the first curved surface 120 is the same in curvature as the curved surface 320 of the light transmission member 300. This is to allow the first curved surface 120 to be entirely in surface contact with the curved surface 320 of the light transmission member 300. It is preferable that the first curved surface 120 is shaped to be convex downwardly of the unit substrate 100. Therefore, as illustrated in FIG. 1, the optical device package 10 is bonded to the light transmission member 300 so as to easily come into surface contact with the outer surface of the light transmission member 300.

The first curved surface 120 is formed on any one of the surface parallel to the X-axis of the unit substrate 100 and the surface parallel to the Y-axis thereof. In FIGS. 2, 3A, and 3B, the first curved surface 120 is formed on the surface parallel to the Y-axis of the unit substrate 100. The provision of the first curved surface 120 ensures that when the optical device package 10 is provided on the light transmission member 300 having a substantially cylindrical shape as illustrated in FIG. 1, the surface contact between the first curved surface 120 and the light transmission member 300 is more easily achieved.

It is preferable that the center point of the first curved surface 120 is coaxially located with the center point of the unit substrate 100. Furthermore, it is preferable that the center point of the cavity 110 is located on the center point of the first curved surface 120.

The first curved surface 120 and the cavity 110 are formed to have the above-described formation positions, whereby the cavity 110 is located at the center of the unit substrate 100, and also the lowest point of the first curved surface 120 is located on the center point of at least one of the X-axis and the Y-axis of the unit substrate 100.

The above-described configuration ensures that even if the multiple optical device packages 10 are provided on the light transmission member 300, when the ultraviolet optical device 200 is mounted at the center of the cavity 110 of each of the optical device packages 10, light emitted from the ultraviolet optical devices 200 mounted on the optical device packages 10 is emitted such that the directions of the light are all toward the central axis of the light transmission member 300.

The ultraviolet optical device 200 is mounted in the cavity 110, and the ultraviolet optical device 200 mounted in the cavity 110 is illuminated in response to application of voltage, thus functioning as a light emitter.

The ultraviolet optical device 200 may be a conventional light emitting diode (LED), but the type of the ultraviolet optical device 200 is not limited thereto.

The ultraviolet optical device 200 is located on the second metal substrate 102, whereby voltage applied to the second metal substrate 102 is directly applied to the ultraviolet optical device 200 being in contact with the second metal substrate 102.

The ultraviolet optical device 200 is connected to the first metal substrate 101 through a wire 210, whereby voltage applied to the first metal substrate 101 is applied to the ultraviolet optical device 200.

The above-described configuration ensures that when voltages of different polarities are applied to the first and second metal substrates 101 and 102, the voltages of different polarities are also applied to the ultraviolet optical device 200, whereby the ultraviolet optical device 200 emits light.

For example, when a positive (+) voltage is applied to the first metal substrate 101 and a negative (−) voltage is applied to the second metal substrate 102, the positive (+) voltage is applied to the ultraviolet optical device 200 through the wire 210 and the negative (−) voltage is applied to the ultraviolet optical device 200 through direct connection between the second metal substrate 102 and the ultraviolet optical device 200.

The first curved surface 120 of the optical device package 10 is configured such that a metal surface of the first metal substrate 101 or the second metal substrate 102 remains exposed, or a separate metal plated layer is formed.

The cavity 110 is provided with a metal pad for facilitating bonding of the ultraviolet optical device 200 on a bottom surface thereof on which the ultraviolet optical device 200 is mounted.

The ultraviolet sterilization module 1 having the above-described configuration ensures that ultraviolet light emitted from the ultraviolet optical devices 200 is radiated toward the flow passage 310 of the light transmission member 300, thus achieving sterilization of the fluid flowing through the flow passage 310.

The ultraviolet sterilization module 1 according to the first embodiment of the present invention having the above-described configuration has the following effects.

The first curved surface 120 is formed on the upper surface of the unit substrate 100, whereby the upper surface has a shape corresponding to the shape of the curved surface 320 of the light transmission member 320. This makes it possible for the light transmission member 300 and the optical device package 10 to be bonded together such that the optical device package 10 comes into surface contact with the light transmission member 300. Therefore, it is possible to prevent defective bonding, leakage of light, and the like that may be caused when a portion of the optical device package 10 is not in contact with the light transmission member 300.

The ultraviolet sterilization module 10 according to the first embodiment of the present invention has a configuration in which provision of a light transmission member bonded to the upper surface of an optical device package of an ultraviolet sterilization module according to the related art is eliminated, and the surface (that is, the upper surface of the optical device package 10) that is to come into direct contact with the light transmission member 300 having the curved surface 320 is changed to the first curved surface 120. Therefore, it is possible to prevent the transmittance from being decreased due to the light transmission member of the ultraviolet sterilization module according to the related art when light generated in the optical device is radiated.

Described in detail, in the case where the light transmission member 300 is made of quartz, the transmittance of the ultraviolet light radiated from the ultraviolet optical device 200 is decreased by the transmittance of the light transmission member. As in the related art, in the case where a separate light transmission member is bonded to the optical device package, and a light transmission member having a curved surface is further bonded to the upper surface of the light transmission member, ultraviolet light has to pass though two light transmission members, leading to a significant decrease in transmittance of ultraviolet light. However, in the case of the ultraviolet sterilization module 1 according to the first embodiment of the present invention, the curved surface 320 of the light transmission member 300 and the first curved surface 120 of the optical device package 10 come into surface contact with each other, thus eliminating requirement of a separate light transmission member to be bonded. This makes it possible to prevent the transmittance of ultraviolet light generated in the ultraviolet optical device 200 from being decreased.

Furthermore, due to the fact that the curved surface 320 of the light transmission member 300 and the first curved surface 120 come into surface contact with each other, the light transmission member 300 and the optical device package 10 are bonded together in close contact with each other. This makes it possible to effectively prevent foreign substances such as water or the like from penetrating into the cavity 110. Therefore, the present invention provides an excellent effect in terms of protection of the ultraviolet optical device 200 mounted on the optical device package 10.

The unit substrate 100 of the optical device package 10 may have a second curved surface formed on a lower surface thereof and having the same curvature as that of the first curved surface 120.

Due to the fact that the second curved surface is formed as described above, the unit substrate 100 of the optical device package 10 is disposed on the light transmission member 300 at the same curvature as that of the light transmission member 300. Therefore, when an electrode part is connected to the lower surface of the unit substrate 100, the electrode part is easily connected to the optical device package 10 by providing the electrode part having an overall curved shape.

In other words, the light transmission member 300, the upper surface of the unit substrate 100 (that is, the upper surface of the unit substrate 100), and the lower surface of the optical device package 10 (that is, the lower surface of the unit substrate 100) all have a curved shape with a predetermined curvature. Therefore, when the optical device package 10, the light transmission member 300, and the electrode part are all combined, it is possible to easily maintain the overall shape of the ultraviolet sterilization module 1 in a curved shape.

As illustrated in FIG. 1, it is preferable that the vertical insulating layer 103 of the optical device package 10 of the ultraviolet sterilization module 1 is provided such that a longitudinal direction thereof is perpendicular to the central axis of the light transmission member 300, and the first curved surface 120 is provided on the surface parallel to the longitudinal direction of the vertical insulating layer 103, that is, the surface parallel to the Y-axis direction in FIGS. 2, 3A, and 3B.

Due to the fact that the vertical insulating layer 103 and the first curved surface 120 have the above configurations, as illustrated in FIG. 1, the first metal substrate 101 is located forwardly of the light transmission member 300 and the second metal substrate 102 is located rearwardly of the light transmission member 300 with respect to the vertical insulating layer 103. In other words, the first and second metal substrates 101 and 102 of the optical device package 10 are disposed on the light transmission member 300 in respective predetermined orientations.

Therefore, it is possible to easily apply different voltages to the first and second metal substrates 101 and 102 through an electrode part. For example, when a positive (+) voltage is applied to the first metal substrates 101 and a negative (−) voltage is applied to the second metal substrates 102, a portion of the electrode part from which the positive (+) voltage is applied is located forwardly of the light transmission member 300 with respect to the vertical insulating layer 103, and a portion of the electrode part from which the negative (−) voltage is applied is located rearwardly of the light transmission member 300 with respect to the vertical insulating layer 103.

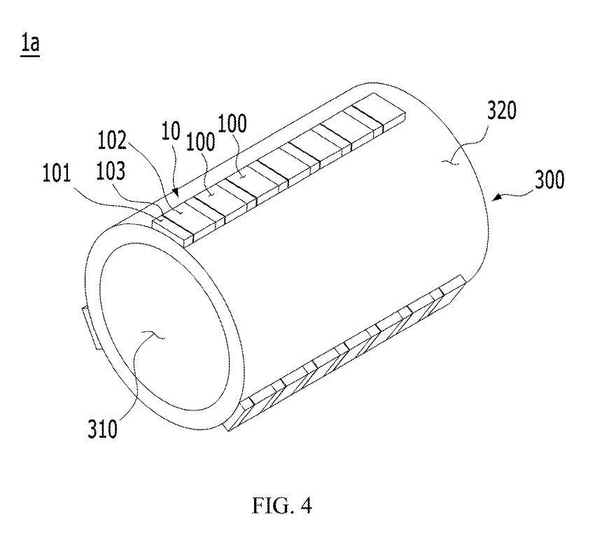

The ultraviolet sterilization module 1 according to the first embodiment of the present invention described above may have other modified embodiments, and an ultraviolet sterilization module 1a according a modified embodiment is illustrated in FIG. 4.

FIG. 4 is a perspective view illustrating a modified embodiment of the ultraviolet sterilization module according to the first embodiment of the present invention.

The ultraviolet sterilization module 1a of FIG. 4 is configured such that an optical device package 10 is bonded to an outer surface of a light transmission member 300 in a state where multiple unit substrates 100 are connected to each other along a longitudinal direction of the light transmission member 300 (or a central axis direction of the light transmission member 300).

In this case, a metal substrate that is constituted by the multiple unit substrates 100 connected to each other is used without cutting such that the multiple unit substrates 100 remain connected to each other in a left-to-right direction, that is, in the X-axis direction in FIG. 2. In this state, the optical device package 10 is bonded to the outer surface of the light transmission member 300.

Multiple optical device packages 10 each having the multiple unit substrates 100 connected to each other in the longitudinal direction of the light transmission member 300 are provided. As illustrated in FIG. 4, three optical device packages 10 are bonded to the light transmission member 300, and the three optical device packages 10 are circumferentially arranged on the light transmission member 300 around the central axis thereof at regular angular intervals of 120 degrees.

As described above, due to the fact that the optical device packages 10 of the ultraviolet sterilization module 1a are provided on the light transmission member 300 such that the multiple unit substrates 100 are arranged along the longitudinal direction of the light transmission member 300, it is possible for ultraviolet light to be radiated along a flow direction of a fluid flowing through a flow passage 310. Therefore, it is possible to achieve uniform sterilization of the fluid from a fluid inlet side and to a fluid outlet side.

In the case of the ultraviolet sterilization module 1a described above, as illustrated in FIG. 4, it is preferable that vertical insulating layers 103 of each of the multiple optical device packages 10 are provided such that longitudinal directions thereof are perpendicular to the central axis of the light transmission member 300, and first curved surfaces 120 are provided on the surfaces parallel to the longitudinal directions of the vertical insulating layers 103, that is, the surfaces parallel to the Y-axis direction in FIGS. 2, 3A, and 3B.

Due to the fact that the vertical insulating layers 103 and the first curved surfaces 120 have the above configurations, as illustrated in FIG. 4, first metal substrates 101 are located either forwardly or rearwardly of the light transmission member 300 and second metal substrates 102 are located in the other direction of the light transmission member 300 with respect to the vertical insulating layers 103.

In other words, the first and second metal substrates 101 and 102 of the optical device package 10 are disposed on the light transmission member 300 in respective predetermined to orientations. Therefore, it is possible to easily apply different voltages to the first and second metal substrates 101 and 102 through an electrode part.

Ultraviolet Sterilization Module 1′ According to a Second Embodiment of the Present Invention

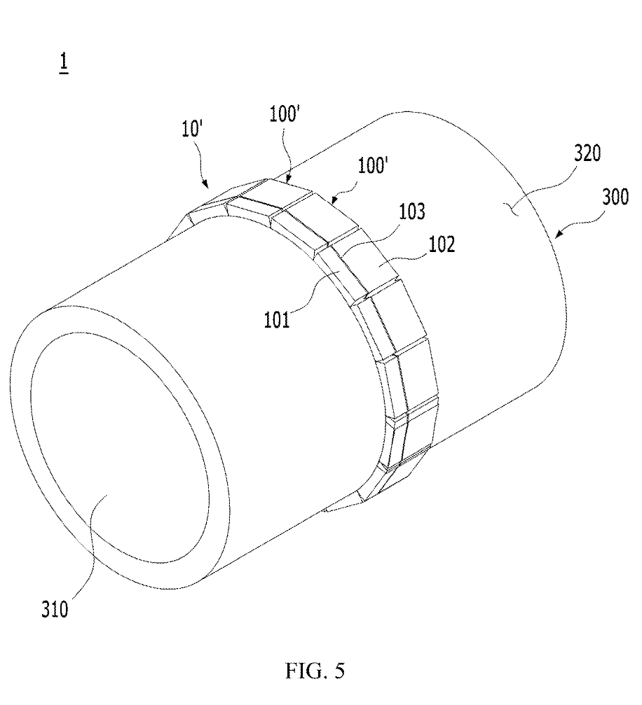

Hereinbelow, an ultraviolet sterilization module 1′ according to a second embodiment of the present invention will be described with reference to FIGS. 5 to 7B.

FIG. 5 is a perspective view illustrating an ultraviolet sterilization module according to a second embodiment of the present invention, FIG. 6 is a perspective view illustrating that an optical device is mounted on an optical device package of FIG. 5, FIG. 7A is a sectional view taken along an X-axis of the optical device package of FIG. 6, and FIG. 7B is a sectional view taken along a Y-axis of the optical device package of FIG. 6.

As illustrated in FIG. 5, the ultraviolet sterilization module 1′ according to the second embodiment of the present invention includes a light transmission member 300 having a flow passage 310 that is formed therein and through which a fluid flows, and an optical device package 10′ provided on an outer surface of the light transmission member 300.

Furthermore, the optical device package 10′ includes: a unit substrate 100′ having first and second metal substrates 101 and 102 that are bonded together on side surfaces thereof in a horizontal direction, and a vertical insulating layer 103 provided between the first and second metal substrates 101 and 102 so as to electrically insulate the first and second metal substrates 101 and 102 from each other; a cavity 110 provided in an upper surface of the unit substrate 100′; a first curved surface 120 provided on the upper surface of the unit substrate 100; and an ultraviolet optical device 200 mounted in the cavity 110. In this case, the first curved surface 120 of the unit substrate 100′ of the optical device package 10′ comes into surface contact with the outer surface of the light transmission member 300.

Furthermore, the optical device package 10′ is provided on an outer circumferential surface of the light transmission member 300 having a cylindrical shape in a state where multiple unit substrates 100′ are connected to each other by junctions 140. Each of the junctions 140 has a groove 150 formed in a lower surface of the unit substrate 100′ to be open downward.

The ultraviolet sterilization module 1′ according to the second embodiment of the present invention differs from the ultraviolet sterilization module 1 according to the first embodiment only in that the optical device package 10′ is constituted by the multiple unit substrates 100′, and the multiple unit substrates 100′ of the optical device package 10′ are connected to each other by the junctions 140, but the remaining components are the same. Therefore, a repetitive description of the same components will be omitted.

The multiple unit substrates 100′ of the optical device package 10′ are connected to each other by the junctions 140, and the junctions 140 have the grooves 150 formed at lower sides thereof, that is, in a lower surface of the optical device package 10′.

Due to the fact that the grooves 150 are formed in the lower surface of the optical device package 10′, the grooves 150 function to facilitate rolling of the multiple unit substrates 100′ when the unit substrates 100′ are rolled as illustrated in FIGS. 4 and 6. In this case, it is preferable that the grooves 150 are formed in directions perpendicular to first curved surfaces 120.

Referring to FIGS. 7A and 7B, the first curved surfaces 120 are formed parallel to the Y-axis direction as illustrated in FIG. 7B.

On the other hand, the grooves 150 are configured such that longitudinal directions thereof are parallel to the X-axis direction as illustrated in FIG. 4.

Due to the fact that the first curved surfaces 120 and the grooves 150 are provided in directions perpendicular to each other as described above, as illustrated in FIG. 6, when the multiple unit substrates 100′ are bonded to the outer surface of the light transmission member 300 by being arranged along a circumferential direction thereof, the grooves 150 easily perform the function of facilitating the rolling of the multiple unit substrates 100′.

Hereinbelow, one example of a manufacturing method of the optical device package 10′ will be described.

It is to be noted that a metal substrate referred to in the following description is a substrate before unit substrates 100′ are cut or divided into predetermined units, that is, a substrate in which multiple first and second metal substrates 101 and 102 that are bonded together by vertical insulating layers 103 are arranged. In this case, a direction from the left side to the right side of the metal substrate is an X-axis direction, and a direction from the front side to the rear side is a Y-axis direction.

First, the metal substrate having the multiple first and second metal substrates 101 and 102 bonded together by the vertical insulating layers 103 is prepared. In this case, the vertical insulating layers 103 are configured such that longitudinal directions thereof are parallel to the X axis. In other words, the first and second metal substrates 101 and 102 are bonded to each other in a front-and-rear direction with the vertical insulating layers interposed between the first and second metal substrates 101 and 102 bonded together.

Thereafter, an upper surface of the metal substrate is formed to have first curved surfaces 120 having the same curvature as that of a curved surface 320 of a light transmission member 300. In this case, the number of the first curved surfaces 120 is equal to the number of the unit substrates 100′ each constituted by bonding of a first metal substrate 101, a second metal substrate 102, and a vertical insulating layer 103.

The upper surface of the metal substrate is formed to have cavities 110 so as to be located at the centers of the first curved surfaces 120.

Furthermore, a lower surface of the metal substrate is formed to have grooves 150. In this case, the grooves 150 are formed to define boundaries between the unit substrates 100′.

The grooves 150 are formed such that the multiple unit substrates 100′ remain connected to each other. Therefore, junctions 140 are formed at upper sides of the grooves 150.

After the first curved surfaces 120, the cavities 110, and the grooves 150 are formed on the metal substrate, the metal substrate is cut along a plane perpendicular to the Y-axis. Thereafter, forming of the optical device package 10′ as illustrated in FIG. 6 is completed.

Furthermore, before cutting the metal substrate, an ultraviolet optical device 200 may be mounted on each of the cavities 110 of one unit substrate 100′, and a wire (not shown) may be connected thereto.

As illustrated in FIG. 5, it is preferable that the vertical insulating layers 103 of the optical device package 10′ of the ultraviolet sterilization module 1 are provided such that longitudinal directions thereof are perpendicular to the central axis of the light transmission member 300 and the first curved surfaces 120 are provided on the surfaces parallel to the longitudinal directions of the vertical insulating layers 103, that is, the surfaces parallel to the Y-axis direction in FIG. 7.

Due to the fact that the vertical insulating layers 103 and the first curved surfaces 120 have the above configurations, as illustrated in FIG. 5, each of the first metal substrates 101 is located forwardly of the light transmission member 300 and each of the second metal substrates 102 is located rearwardly of the light transmission member 300 with respect to the vertical insulating layer 103. In other words, the first and second metal substrates 101 and 102 of the optical device package 10′ are disposed on the light transmission member 300 in respective predetermined orientations.

Therefore, it is possible to easily apply different voltages to the first and second metal substrates 101 and 102 through an electrode part. For example, when a positive (+) voltage is applied to the first metal substrates 101 and a negative (−) voltage is applied to the second metal substrates 102, a portion of the electrode part from which the positive (+) voltage is applied is located forwardly of the light transmission member 300 with respect to the vertical insulating layers 103, and a portion of the electrode part from which the negative (−) voltage is applied is located rearwardly of the light transmission member 300 with respect to the vertical insulating layers 103.

Furthermore, as described above, due to the fact that the vertical insulating layers 103 are provided such that the longitudinal directions thereof are perpendicular to the central axis of the light transmission member 300 and the first curved surfaces 120 are provided on the surfaces parallel to the longitudinal directions of the vertical insulating layers 103, that is, the surfaces parallel to the Y-axis direction, the first and second metal substrates 101 and 102 of the multiple unit substrates 100′ of the optical device package 10′ are arranged on the light transmission member 300 in respective predetermined orientations, whereby the multiple unit substrates 100′ of the optical device package 10′ are connected to each other in parallel.

Unlike the above description, the vertical insulating layers 103 of the optical device package 10′ may be provided such that the longitudinal directions thereof are parallel to the central axis of the light transmission member 300 and the first curved surfaces 120 are provided on the surfaces perpendicular to the longitudinal directions of the vertical insulating layers 103, that is, the surfaces parallel to the X-axis direction. In this case, the multiple unit substrates 100′ of the optical device package 10′ may be connected to each other in series.

The ultraviolet sterilization module 1′ according to the second embodiment of the present invention having the above-described configuration has the following effects in addition to the effects of the ultraviolet sterilization module 1 according to the first embodiment described above.

When the optical device package 10′ is provided on the curved surface 320 of the light transmission member 300, the multiple unit substrates 100′ are connected to each other by the junctions 140, while the grooves 150 help the optical device package 10′ rolled in an overall circular shape to correspond to the shape of the curved surface 320 of the light transmission member 300. Therefore, the multiple unit substrates 100′ are bonded to the light transmission member 300 in surface contact with the curved surface 320 of the light transmission member 300 in a state of being connected to each other. This makes it possible to bond a large number of unit substrates 100′ to the light transmission member 300 as compared with the first embodiment of the present invention. Therefore, the amount of ultraviolet light radiated from the ultraviolet optical devices 200 is increased, thus achieving an increase in sterilization efficiency of the ultraviolet sterilization module 1′.

Furthermore, due to the fact that the multiple unit substrates 100′ of the optical device package 10′ are connected to each other, it is possible to achieve a strong bonding with the light transmission member 300, thus preventing the ultraviolet sterilization module 1′ from being damaged.

In the optical device package 10′ of the ultraviolet sterilization module 1′ according to the second embodiment of the present invention, each of the multiple unit substrates 100′ has a second curved surface formed on a lower surface thereof like ultraviolet sterilization module 1 according to the first embodiment of the present invention, and a description thereof can be substituted for the above and thus will be omitted.

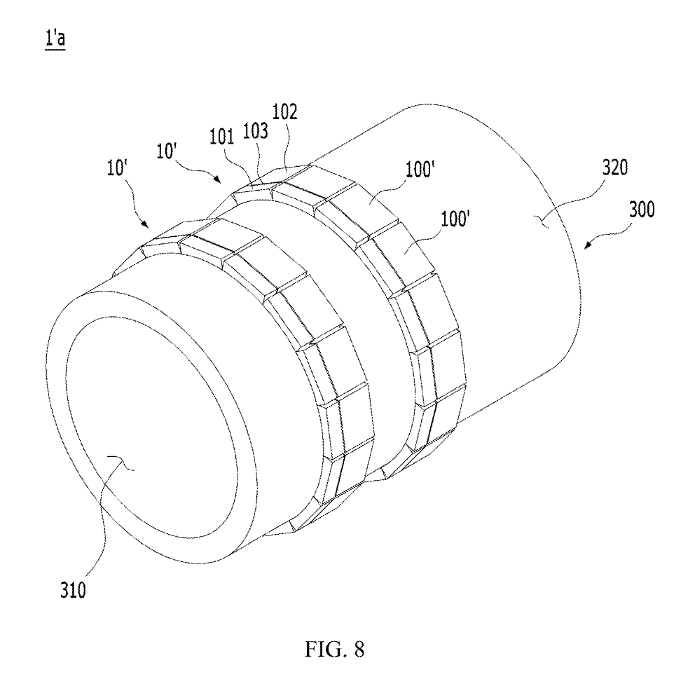

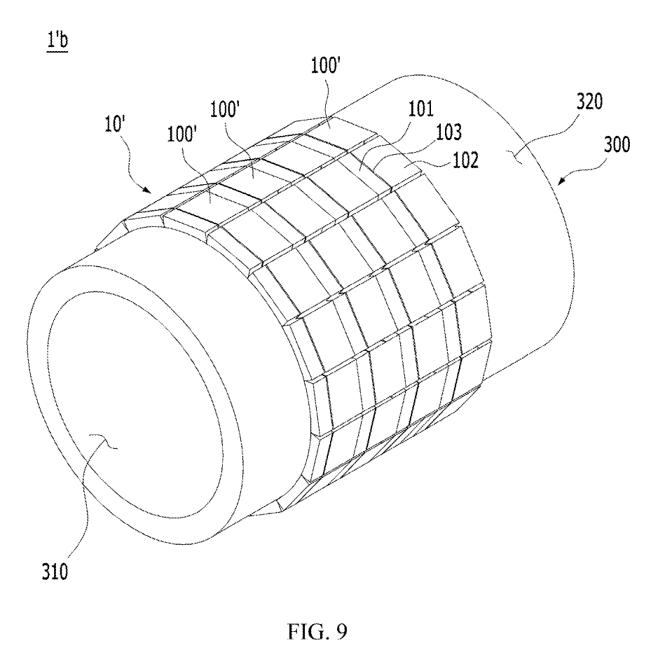

The ultraviolet sterilization module 1′ according to the second embodiment of the present invention described above may have other modified embodiments. An ultraviolet sterilization module 1′a according to a first modified embodiment is illustrated in FIG. 8, and an ultraviolet sterilization module 1′b according to a second modified embodiment is illustrated in FIG. 9.

First, the ultraviolet sterilization module 1′a according to the first modified embodiment will be described with reference to FIG. 8.

FIG. 8 is a perspective view illustrating the first modified embodiment of the ultraviolet sterilization module according to the second embodiment of the present invention.

As illustrated in FIG. 8, an optical device package 10′ of the ultraviolet sterilization module 1′a is bonded to an outer circumferential surface a light transmission member 300 in a state where multiple unit substrates 100′ are connected to each other. Multiple optical device packages 10′ (in FIG. 8, two optical device packages 10′) are bonded to the light transmission member 300 along a longitudinal direction thereof.

As described above, due to the fact that the multiple optical device packages 10′ are bonded to the outer circumferential surface of the light transmission member 300 along the longitudinal direction of the light transmission member 300, it is possible to radiate a greater amount of ultraviolet light along a flow direction of a fluid flowing through a flow passage 310, thus further improving an effect of sterilizing the fluid.

Hereinbelow, the ultraviolet sterilization module 1′b according to the second modified embodiment will be described with reference to FIG. 9.

FIG. 9 is a perspective view illustrating the second modified embodiment of the ultraviolet sterilization module according to the second embodiment of the present invention.

An optical device package 10′ of the ultraviolet sterilization module 1b illustrated in FIG. 9 is bonded to an outer circumferential surface of a light transmission member 300 in a state where multiple unit substrates 100′ are connected to each other. Described in detail, the multiple unit substrates 100′ of the optical device package 10′ are bonded to the light transmission member 300 in a state of being connected to each other along circumferential and longitudinal directions of the light transmission member 300. In this case, no grooves 150 are formed between the multiple unit substrates 100′ connected to each other along the longitudinal direction of the light transmission member 300.

The optical device package 10′ is manufactured by the above-described manufacturing method in a manner that first curved surfaces 120, cavities 110, and grooves 150 are formed on a metal substrate, a portion of the metal substrate is cut along a plane perpendicular to the Y-axis, and a remaining portion of the metal substrate is not cut along the plane perpendicular to the Y-axis.

In other words, the optical device package 10′ is provided on the light transmission member 300 such that the number of the remaining portions of the metal substrate that remain without cutting is equal to the number of the unit substrates 100′ provided on the light transmission member 300 along the longitudinal direction thereof.

The ultraviolet sterilization module 1b ensures that multiple ultraviolet optical devices 200 radiate ultraviolet light in a direction in which the fluid flows through the flow passage 310, and also the unit substrates 100′ are bonded to the light transmission member 300 along the longitudinal direction thereof in a dense arrangement without wasting space. Therefore, it is possible to significantly increase the amount of ultraviolet irradiation per same area, thus significantly improving an effect of sterilizing the fluid.

As described above, the ultraviolet sterilization module 1′a according to the first modified embodiment and the ultraviolet sterilization module 1b according to the second modified embodiment provide an increased amount of ultraviolet irradiation per same area, and which is advantageous over the ultraviolet sterilization module 1′ according to the second embodiment. This makes it possible to improve a sterilization effect without requiring provision of a large ultraviolet sterilization module. In other words, through a change in arrangement structure of the optical device package, it is possible to achieve a size reduction of the ultraviolet sterilization module.

Ultraviolet Sterilization Module 1″ According to a Third Embodiment of the Present Invention

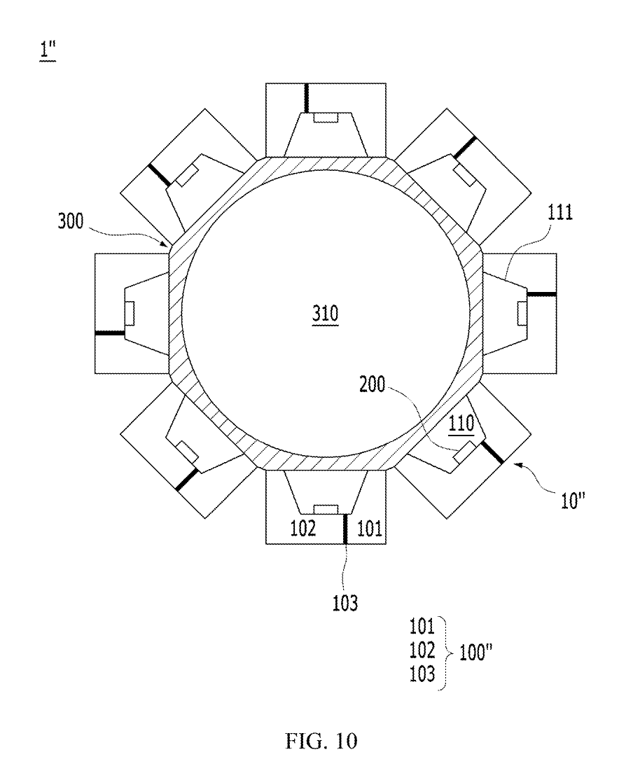

Hereinbelow, an ultraviolet sterilization module 1″ according to a third embodiment of the present invention will be described with reference to FIG. 10.

As illustrated in FIG. 10, the ultraviolet sterilization module 1″ according to the third embodiment of the present invention includes a light transmission member 300 having a flow passage 310 that is formed therein and through which a fluid flows, and multiple optical device packages 10″ provided on an outer surface of the light transmission member 300. Each of the multiple optical device packages 10″ includes: a unit substrate 100″ having first and second metal substrates 101 and 102 that are bonded together on side surfaces thereof in a horizontal direction, and a vertical insulating layer 103 provided between the first and second metal substrates 101 and 102 so as to electrically insulate the first and second metal substrates 101 and 102 from each other; a cavity 110 provided in an upper surface of the unit substrate 100″; and an ultraviolet optical device 200 mounted in the cavity 110, wherein the light transmission member 300 has a substantially prism shape.

In other words, the ultraviolet sterilization module 1″ according to the third embodiment of the present invention differs from the ultraviolet sterilization module 1 according to the first embodiment only in that a first curved surface 120 is not provided on the upper surface of the unit substrate 100″ of the optical device package 10″ and the light transmission member 300 has a prism shape, but the remaining components are the same. Therefore, a repetitive description of the same components will be omitted.

In the ultraviolet sterilization module 1″ according to the third embodiment of the present invention having the above-described configuration, the outer surface of the light transmission member 300 is polygonal in cross-section. This ensures that even when the upper surface of the optical device package 10″ is not provided with the curved surface, the upper surface of the optical device package 10″ and the light transmission member 300 come into surface contact with each other.

Therefore, it is possible for ultraviolet light to be radiated from the outside to inside of the light transmission member 300 through the ultraviolet optical devices 200 even without performing a process for forming the curved surface.

Furthermore, the upper surface of the optical device package 10″ and the outer surface of the light transmission member 300 come into close surface contact with each other, thus eliminating requirement of a separate light transmission member. This makes it possible to prevent a decrease in transmittance of ultraviolet light and penetration of foreign substances into the cavity 110.

As described above, the present invention has been described with reference to the exemplary embodiments. However, those skilled in the art will appreciate that various modifications, additions and substitutions are possible, without departing from the scope and spirit of the invention as disclosed in the accompanying claims.

What is claimed is:

1. An ultraviolet sterilization module, comprising:
a light transmission member having a flow passage that is formed therein and through which a fluid flows; and
an optical device package provided on an outer surface of the light transmission member,
wherein the optical device package includes:
a unit substrate including first and second metal substrates that are bonded together on side surfaces thereof in a horizontal direction, and a vertical insulating layer provided between the first and second metal substrates so as to electrically insulate the first and second metal substrates;
a cavity provided in an upper surface of the unit substrate; and
an ultraviolet optical device mounted in the cavity,
wherein an upper surface of the unit substrate is in surface contact with the outer surface of the light transmission member.

2. The ultraviolet sterilization module of claim 1, wherein the light transmission member has a cylindrical shape, and
the unit substrate of the optical device package has a first curved surface having the same curvature as a curvature of the cylindrical-shaped light transmission member.

3. The ultraviolet sterilization module of claim 2, wherein the optical device package is provided on an outer circumferential surface of the cylindrical-shaped light transmission member in a state where multiple unit substrates are connected to each other by junctions.

4. The ultraviolet sterilization module of claim 3, wherein each of the junctions has a groove formed in a lower surface of the unit substrate to be open downward.

5. The ultraviolet sterilization module of claim 3, wherein the vertical insulating layer of the optical device package is provided such that a longitudinal direction thereof is perpendicular to a central axis of the light transmission member.

6. The ultraviolet sterilization module of claim 1, wherein the optical device package is provided in plural, and multiple optical device packages are configured such that multiple unit substrates of each of the optical device packages are connected to each other and arranged along a longitudinal direction of the cylindrical-shaped light transmission member.

7. The ultraviolet sterilization module of claim 6, wherein the vertical insulating layers of the optical device packages are provided such that longitudinal directions thereof are parallel to a central axis of the light transmission member.
